(19) United States
(12) Patent Application Publication
(10) Pub. No.: US 2026/0249003 A1
(43) Pub. Date: Aug. 27, 2026

(54) INSULIN SYRINGE REPLACEMENT SYSTEM FOR INSULIN PUMP

(71) Applicant: G2E CO., LTD, Seoul (KR)

(72) Inventor: Dong Hun LEE, Bucheon-si (KR)

(21) Appl. No.: 19/644,411

(22) Filed: Apr. 10, 2026

Related U.S. Application Data
(63) Continuation of application No. PCT/KR2023/015582, filed on Oct. 11, 2023.

(51) Int. Cl. A61M 5/172; A61M 5/142

(52) U.S. Cl. CPC A61M 5/172; A61M 5/14248; A61M 2205/14; A61M 2205/3317; A61M 2205/6018; A61M 2205/6054; A61M 2205/8206

(57) ABSTRACT

The disclosed relates to an insulin syringe replacement system for an insulin pump. The system comprises: a control unit for controlling the operation of the insulin pump; an operation unit for performing an initialization setting operation when an insulin syringe is replaced under the control of the control unit; and a battery unit for supplying power to the control unit and the operation unit; a housing unit for mounting the insulin syringe; and a sensor unit for confirming whether a stopper of the insulin syringe mounted on the housing unit is coupled and notifying the control unit of the confirmation, wherein the control unit controls the operation unit to perform the initialization setting operation only after receiving the confirmation from the sensor unit that the stopper of the insulin syringe mounted on the housing unit is coupled.

### TECHNICAL FIELD

The present disclosure relates to an insulin pump that is a medical device for diabetic patients, and more particularly, to a system for providing a safety device to prevent accidents that may occur when replacing an insulin syringe that supplies insulin to an insulin pump.

### BACKGROUND ART

Diabetes is a condition that happens when the blood sugar level stays high for a prolonged time due to the human body's inability to normally maintain the hormone secretion system for maintaining the blood sugar level, causing excretion of glucose in the patient's urine. The high urine glucose level leads to increased urination, dehydration and intense thirst. Furthermore, the body is unable to use nutrients, and nutrient loss causes chronic fatigue. In addition, severe diabetes causes damage to the blood vessels in which high-glucose blood flows, causing impaired blood supply, and it may damage retina, kidneys and nerves, leading to vision loss, chronic renal failure or numbness and pain in the upper and lower extremities.

Diabetes treatment may include diet, exercise and medication. Here, the medication is used to treat diabetes by injecting insulin into the body by various methods, based on the understanding that diabetes is caused by a deficiency of hormone insulin that maintains the appropriate blood sugar level. The exogenous insulin administered to replace the function of the pancreas lowers and maintains the patient's blood sugar level to the normal level and alleviates the above-described symptoms of diabetes.

To properly inject insulin into the patient's body, insulin pumps are widely used. An insulin pump generally has a size that is easy to carry and is connected to the patient's body through a tube and a needle. The insulin pump is always connected and carried by the patient, and is designed to automatically control the dosage using electronic and mechanical devices.

Meanwhile, insulin administration to diabetic patients is done in precise units. This is because even a very small amount of insulin may significantly change the patient's blood sugar level. For this reason, the insulin pump is designed to deliver insulin in such a way that multiple gear devices rotate to slightly move a plunger movement rod forward to force the plunger movement rod to push a plunger gasket of an insulin syringe. A control unit of the insulin pump determines an amount of insulin to be administered by determining the number of revolutions of the gear devices. FIG. 1 shows the insulin pump with the above-described system.

Meanwhile, for the above-described tasks, the insulin pump is required to pre-identify the exact amount of insulin stored in the insulin syringe. To do so, a task performed by the insulin pump is hereinafter referred to as ‘initialization setting operation’. The initialization setting operation is an operation in which the gear devices rotate to move the plunger movement rod forward until the plunger movement rod contacts the plunger gasket, and the control unit measures the number of revolutions of the gear devices during the initialization setting operation to determine the exact amount of insulin stored in the insulin syringe. During the initialization setting operation, the plunger movement rod stops moving forward when a repulsive force of a predetermined strength or more is detected through a pressure detection sensor. The repulsive force is generated by the increased pressure in the cylinder when the outlet of the insulin syringe is closed with a cap to prevent insulin leaks. Accordingly, to perform the normal initialization setting operation, it is necessary that a user directly closes the outlet with the cap beforehand.

This task is generally performed by the patient himself/herself, and when considering the age and physical condition of the diabetic patient, the patient often skips the above-described essential pre-task before the start of the initialization setting process of the insulin pump. Furthermore, a manipulation device for the insulin pump is generally carried by being attached to the patient's body, so the initialization setting operation may be unintentionally manipulated to begin by the patient's activities, falls or leaning against a wall.

These situations do not occur very frequently, but risks driven by the situations are significantly high. As described above, because the human body is highly sensitive to insulin, the diabetic patient takes very small doses of insulin through deliberate adjustment. In this circumstance, in a situation where the outlet is not closed with the cap, for example, insulin may be injected into the patient connected to the insulin syringe through the tube, when the initialization setting operation is performed, the plunger movement rod that fails to detect the repulsive force will keep moving forward, and eventually a large amount of insulin is injected into the patient's body all at once. This incident may cause acute hypoglycemia, and in worse cases, death.

### SUMMARY

### Technical Problem

The present disclosure is directed to providing an insulin syringe replacement system that may be applied to the existing operation process of an insulin pump to achieve safe insulin syringe replacement and setting initialization operation of the insulin pump.

### Technical Solution

The present disclosure provides an insulin syringe replacement system for providing a safety device for preventing accidental or unexpected accidents that may occur when replacing an insulin syringe, and the insulin syringe replacement system that may be applied to an insulin pump includes a control unit configured to control an operation of the insulin pump; an actuation unit configured to perform an initialization setting operation when replacing the insulin syringe under the control of the control unit; a battery unit configured to supply power to the control unit and the actuation unit; a housing unit configured to mount the insulin syringe; and a sensor unit configured to detect whether a cap of the insulin syringe mounted in the housing unit is coupled and notify it to the control unit, wherein the insulin syringe that is mounted in the housing unit includes a cylinder to store insulin, an outlet positioned at an end of the cylinder to dispense the insulin, a cap coupled to the outlet to prevent the insulin stored in the cylinder from leaking, a plunger gasket located at an opposite end of the cylinder to change an internal pressure of the cylinder, and a plunger movement rod to push the plunger gasket to move the plunger gasket forward, wherein the initialization setting operation performed by the actuation unit is an operation in which the actuation unit moves the plunger movement rod forward until the plunger movement rod reaches the plunger gasket in a state that the cap is coupled to the outlet of the insulin syringe, and wherein the control unit controls the actuation unit to perform the initialization setting operation only after receiving coupling notification of the cap of the insulin syringe mounted in the housing unit from the sensor unit, and recognizes an amount of insulin contained in the cylinder based on a movement distance or a location of the plunger movement rod after completion of the initialization setting operation performed by the actuation unit.

An embodiment of the present disclosure may provide the insulin syringe replacement system for the insulin pump, wherein the housing unit includes a holder where the outlet of the insulin syringe mounted in the housing unit is situated and exposed to an outside of the insulin pump to allow the coupling of the cap, wherein the sensor unit is a magnetic sensor and is located at the holder to detect the coupling of the outlet and the cap, and wherein the cap that is coupled to the outlet has a magnetic property, and when the cap is coupled to the outlet, the magnetic sensor detects magnetic field lines of the cap and notifies it to the control unit.

Another embodiment of the present disclosure may provide the insulin syringe replacement system for the insulin pump, wherein the cap that is coupled to the outlet has a coupling groove extending from a hole into which the outlet is inserted when coupled, and has a magnetic body attached to an area of a surface facing the magnetic sensor when the coupling with the outlet is normally completed, and wherein the outlet that is coupled with the cap has a coupling protrusion on a side, the coupling protrusion configured to be engaged with the coupling groove of the cap that is coupled to the outlet, and when the normal coupling of the outlet and the cap is done, the area of the surface of the cap facing the magnetic sensor is defined.

### Advantageous Effects

The present disclosure has the following effects.

First, it may be possible to fundamentally prevent insulin overdoses that occur when the initialization setting operation is performed by unintentional manipulation driven by the wearer's activities when the insulin pump is connected to the body through the tube.

Second, it may be possible to prevent the waste of replacement insulin syringes caused by the plunger movement rod moving forward until it completely dispenses the insulin contained within the cylinder due to failing to detect a sufficient repulsive force when the user manipulates to perform the initialization setting operation without realizing that he/she did not couple the cap to the insulin pump.

Third, it may be possible to apply this system for safety while maintaining the existing initialization setting operation of the insulin pump, thereby saving additional research and development costs.

### BRIEF DESCRIPTION OF THE DRAWINGS

FIG. 1 shows an existing insulin pump and insulin syringe to which a system of the present disclosure may be applied.

FIG. 2 shows an insulin syringe replacement system according to an embodiment of the present disclosure.

FIG. 3 shows the coupling of a cap of an embodiment of the present disclosure.

FIG. 4 shows an initialization setting process of the insulin pump according to the present disclosure.

### LIST OF REFERENCE NUMERALS

10: Insulin pump
20: Insulin syringe
21: Cylinder
22: Plunger gasket
23: Outlet
23a: Coupling protrusion
24: Cap
24a: Coupling groove
25: Plunger movement rod, Plunger movement screw
26: Plunger movement locking nut
27: Magnetic body
100: Insulin syringe replacement system
110: Actuation unit
111: Electric motor
112: Gear device
120: Control unit
130: Housing unit
131: Holder
140: Sensor unit, Magnetic sensor
150: Battery unit

### DETAILED DESCRIPTION OF EXEMPLARY EMBODIMENTS

Exemplary embodiments of the present disclosure will be described in further detail, and description of well-known technical features is omitted or briefly described for the sake of brevity.

Hereinafter, the exemplary embodiments of the present disclosure will be described in detail with reference to the accompanying drawings.

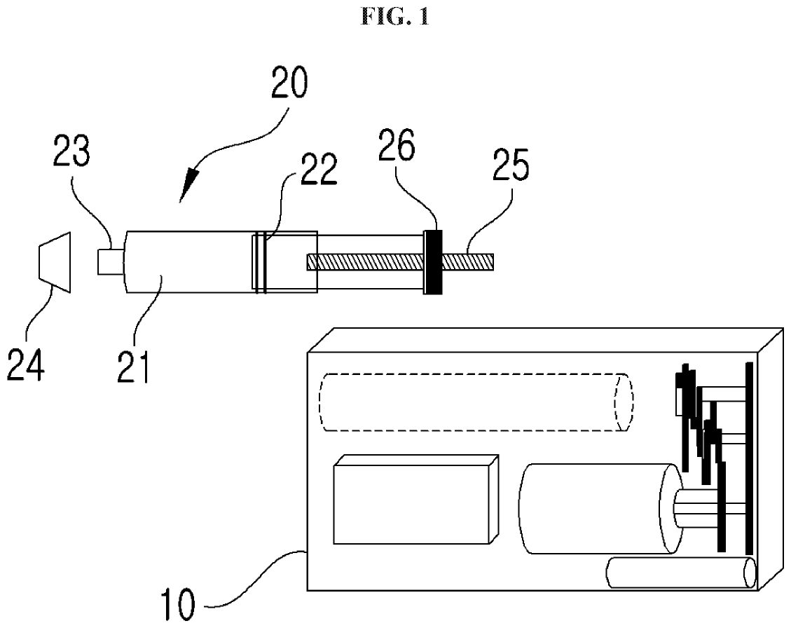

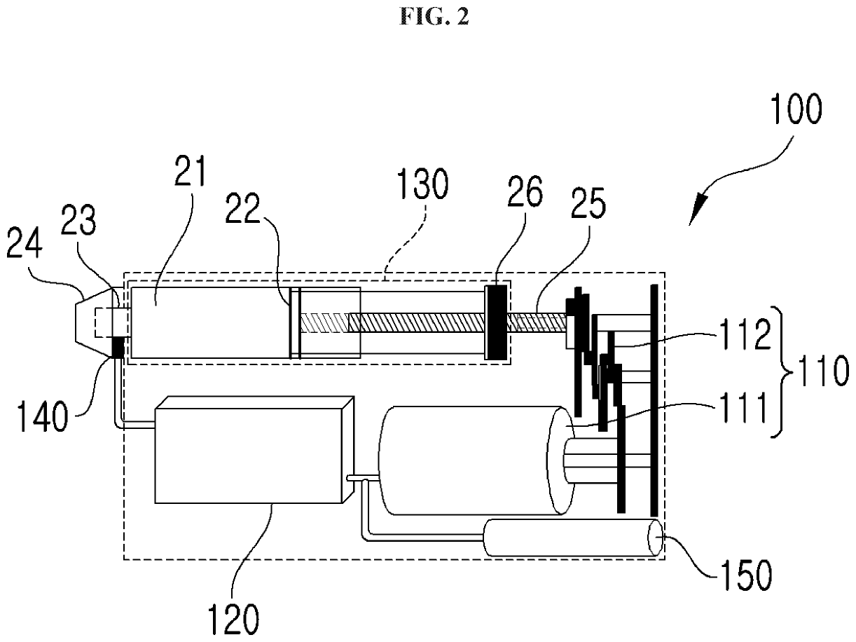

Prior to the description of the present disclosure, it should be noted that an insulin pump 10 to which the system of the present disclosure is applied allows attachment and detachment of a replacement insulin syringe 20 (hereinafter referred to as ‘insulin syringe 20’) to replenish insulin that is administered to a patient. When the insulin pump 10 runs out of insulin, the patient replaces the used insulin syringe 20 with a new insulin syringe 20 and enables the insulin pump 10 to perform an initialization setting operation to measure an amount of insulin stored in the replaced insulin syringe 20. FIG. 1 shows the insulin pump to which the insulin replacement system of the present disclosure is applied, and FIG. 2 shows the operation of the insulin replacement system of the present disclosure. The following description will be made with reference to FIGS. 1 and 2.

The insulin syringe replacement system 100 for the insulin pump 10 according to the present disclosure is formed in the form of the insulin pump 10 including a control unit 120, an actuation unit 110, a battery unit 150, a housing unit 130 and a sensor unit 140. That is, the present disclosure should be understood as a type of system made up of the above-described components of the insulin pump 10.

The control unit 120 is preferably formed in the form of a microchip or a microcontroller that performs calculations for electronically controlling the operation of the insulin pump 10, but may include those that perform the role of the control unit 120 as described above and described below, and it should be understood that they are included in the scope of the present disclosure.

The actuation unit 110 performs the initialization setting operation when replacing the insulin syringe 20 under the control of the control unit 120. The initialization setting operation performed by the actuation unit 110 is an operation in which the actuation unit 110 moves a plunger movement rod 25 forward until it reaches a plunger gasket 22 in a state that a cap 24 is coupled to an outlet 23 of the insulin syringe 20. Accordingly, the actuation unit 110 may be configured to couple with or contact the plunger movement rod 25 to move the plunger movement rod 25 forward. The actuation unit 110 may be configured to move the plunger movement rod 25 forward for not only the initialization setting operation but also a general insulin injection operation.

The actuation unit 110 may include an electric motor to supply a power source for the operation of the actuation unit 110 and a gear device 112 connected to the electric motor 111.

The electric motor may be connected to a battery as described below to receive power from the battery.

The gear device 112 moves the plunger movement rod 25 forward while rotating. The plunger movement rod 25 is generally included as the component of the replacement insulin syringe 20 as described below, but in some cases, may be included in the actuation unit 110 as the component of the insulin syringe replacement system 100 of the present disclosure.

The gear device 112 preferably includes multiple gears having different gear ratios but is not particularly limited thereto. The gear device 112 including multiple gears is easy to perform the operation of injecting a very small amount of insulin.

In an embodiment, the plunger movement rod 25 moving forward by the gear device 112 may be a plunger movement screw 25. In this embodiment, the plunger movement screw 25 is fastened with a plunger movement locking nut 26. The plunger movement locking nut 26 is fixed to the insulin pump 10 or the insulin syringe 20. As the plunger movement locking nut 26 is fixed, when the gear device 112 rotates the plunger movement screw 25 fastened to the plunger movement locking nut 26, the plunger movement screw 25 moves forward. Also in this embodiment, the plunger movement screw 25 and the plunger movement locking nut 26 may be the component of the replacement insulin syringe 20, and may be included in the actuation unit 110 as the component of the insulin syringe replacement system 100 of the present disclosure.

The actuation unit 110 preferably includes a sensor or a device to measure the number of revolutions of the electric motor 111, the gear device 112 or the plunger movement screw 25. The sensor or device provides information associated with the number of revolutions to the control unit 120. The control unit 120 determines the amount of insulin administered based on the received information. To the contrary, the control unit 120 may adjust the angle of rotation of the electric motor 111 to inject a pre-determined amount of insulin. As the gear device 112 including multiple gears having different gear ratios may determine the angle of rotation of the electric motor 111 that is large enough to inject a very small amount of insulin, it may be easier to operate the electric motor 111 to inject a very small amount of insulin.

The battery unit 150 provides power to the control unit 120 and the actuation unit 110.

The housing unit 130 is configured to mount the insulin syringe 20. The insulin pump 10 replenishes insulin by replacing the insulin syringe 20.

The insulin syringe 20 includes a cylinder 21 to store insulin, the outlet 23 positioned at one end of the cylinder 21 to dispense insulin, the cap 24 coupled to the outlet 23 to prevent insulin stored in the cylinder 21 from leaking, the plunger gasket 22 located at the other end of the cylinder 21 to change the internal pressure of the cylinder 21, and the plunger movement rod 25 to push the plunger gasket 22 to move the plunger gasket 22 forward. However, the plunger movement rod 25 may be included in the actuation unit 110 as described above.

When the plunger movement rod 25 is formed in the form of the plunger movement screw 25 as described in the previous embodiment, the insulin syringe 20 further includes the plunger movement locking nut 26. However, also in this case, the plunger movement screw 25 and the plunger movement locking nut 26 may be included in the actuation unit 110. The plunger movement screw 25 may be included in the insulin syringe 20, and the plunger movement locking nut 26 may be included in the actuation unit 110, or vice versa. That is, it should be understood that the plunger movement screw 25 and the plunger movement locking nut 26 may have any configuration for pushing the cylinder 21 to increase pressure or dispense insulin through the outlet 23 by interactions between them when the insulin syringe 20 is mounted in the housing unit 130.

In the initialization setting mode, the cap 24 is coupled to the outlet 23, and accordingly, even though the plunger movement rod 25 pushes the plunger gasket 22, the plunger gasket 22 does not move forward. When the plunger movement rod 25 contacts the plunger gasket 22, pressure is produced by the principle of action and reaction, and when the pressure is detected and notified to the control unit 120, the control unit 120 stops the operation of the actuation unit 110 for moving the plunger movement rod 25 forward.

After the completion of the initialization setting operation performed by the actuation unit 110, the control unit 120 recognizes the amount of insulin contained in the cylinder 21 based on the movement distance or location of the plunger movement rod 25. The movement distance or location of the plunger movement rod 25 may be determined by measuring the angle of rotation of the gears or the electric motor included in the actuation unit 110.

The sensor unit 140 detects whether the cap 24 of the insulin syringe 20 mounted in the housing unit 130 is coupled and notifies it to the control unit 120. The control unit 120 controls the actuation unit 110 to perform the initialization setting operation only after receiving coupling notification of the cap 24 of the insulin syringe 20 mounted in the housing unit 130 from the sensor unit 140.

When the cap 24 is not coupled to the outlet 23, the plunger movement rod 25 keeps pushing the plunger gasket 22 to move the plunger gasket 22 forward after it contacts the plunger gasket 22, and all insulin stored in the cylinder 21 comes out of the outlet 23. When the outlet 23 is connected to the user through the tube, the patient may take an overdose of insulin, leading to death or emergency.

Accordingly, after the sensor unit 140 detects whether the cap 24 is coupled, the initialization setting operation may be performed only after it is detected that the cap 24 is coupled, therefore it is possible to fundamentally prevent accidents that may occur when the initialization setting operation is performed unintentionally or inadvertently.

In another embodiment of the present disclosure, the housing unit 130 may include a holder 131, and the sensor unit 140 may be located at the holder 131. The holder 131 is where the outlet 23 of the insulin syringe 20 mounted in the housing unit 130 is situated and exposed to the outside of the housing unit 130 to allow the coupling of the cap 24. In addition, the sensor unit 140 is located at the holder 131 to easily detect whether the outlet 23 situated at the holder 131 and the cap 24 are coupled together.

In this embodiment, the sensor unit 140 may be a magnetic sensor 140. In this instance, the cap 24 that is coupled to the outlet 23 may have a magnetic property, and when the cap 24 is coupled to the outlet 23, the magnetic sensor 140 may detect magnetic field lines of the cap 24 and notify it to the control unit 120.

In this instance, the magnetic sensor 140 may be preferably located in a direction in which the outlet 23 is exposed to the outside of the insulin pump 10 or the housing unit 130 to easily detect magnetic field lines when the cap 24 is coupled.

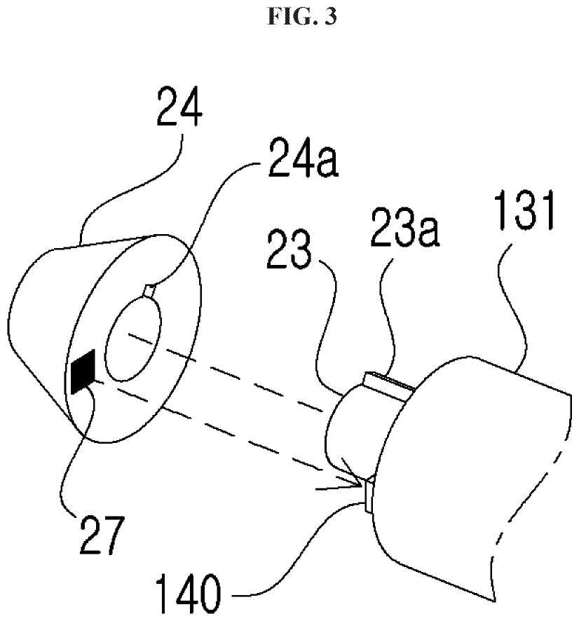

FIG. 3 shows the coupling of the cap 24 of another embodiment of the present disclosure. In this embodiment, a portion of the cap 24 that contacts or is located close to the magnetic sensor 140 on the holder 131 when coupled to the outlet 23 may be made of a magnetic material. In this embodiment, the cap 24 has a coupling groove 24a extending from a hole into which the outlet 23 is inserted, and the outlet 23 has a coupling protrusion 23a that is engaged with the coupling groove 24a on the side. In this case, the cap 24 may be coupled to the outlet 23 in a particular direction, and when normal coupling is done, a particular surface of the cap 24 may face the magnetic sensor 140. In this embodiment, a magnetic body 27 may be attached to the corresponding particular surface, and when the magnetic body 27 contacts or is located close to the magnetic sensor 140, the coupling of the cap 24 may be detected.

To sum up, according to this embodiment, provided is the insulin syringe replacement system in which the cap 24 that is coupled to the outlet 23 has the coupling groove 24a extending from the hole into which the outlet 23 is inserted when coupled, and the cap 24 has the magnetic body 27 attached to the area of the surface facing the magnetic sensor 140 when the coupling with the outlet 23 is normally completed and the outlet 23 that is coupled with the cap 24 has, on the side, the coupling protrusion 23a that is engaged with the coupling groove 24a of the cap 24 that is coupled to the outlet 23, so when normal coupling of the outlet 23 and the cap 24 is done, the area of the surface of the cap 24 facing the magnetic sensor 140 is defined. This embodiment may minimize the use of the magnetic material, thereby reducing the production cost of the cap 24.

Meanwhile, the magnetic sensor 140 is configured to provide coupling notification of the cap 24 to the control unit 120 when sufficiently strong magnetic field lines are detected. The insulin pump 10 is attached to the user's body or clothes, and in some cases, the user's object, for example, clothes, a watch or a mobile phone may have a magnetic property. In this case, unless the magnetic sensor 140 is configured to provide coupling notification of the cap 24 only when sufficiently strong magnetic field lines are detected, even when the cap 24 is not coupled, the magnetic sensor 140 may detect magnetic field lines generated from the user's object and provide coupling notification of the cap 24 to the control unit 120. Accordingly, sufficiently high sensing threshold of the magnetic sensor 140 may be set to prevent errors.

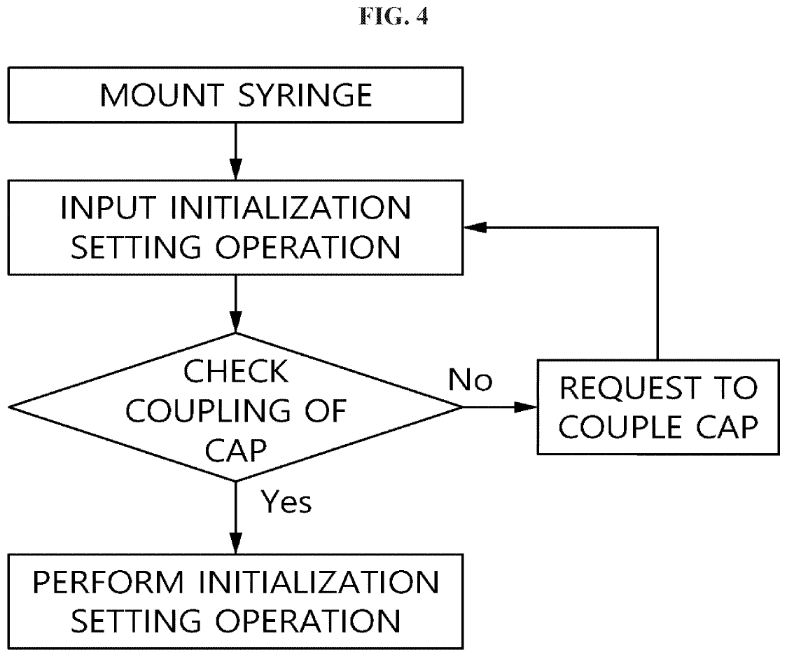

The initialization setting process after the replacement of the insulin syringe 20 of the insulin pump 10 to which the present disclosure is applied will be described with reference to FIG. 4 as follows.

First, the user correctly mounts the syringe in the housing unit 130. Subsequently, the user performs manipulation or input to enable the insulin pump 10 to perform the initialization setting operation. When it receives the input, the control unit 120 checks whether it has received coupling notification of the cap 24 from the sensor unit 140.

When it has received notification, the control unit 120 controls the actuation unit 110 to perform the initialization setting operation.

When it has not received notification, the control unit 120 requests the user to couple the cap 24. A variety of methods may be used, for example, visual (display) or auditory (voice) notification. Subsequently, when the user performs manipulation or input again to perform the initialization setting operation, the control unit 120 checks the coupling of the cap 24 again.

Depending on the presence or absence of coupling notification of the cap 24, the cap 24 goes into any one of the two routes again.

Although the embodiments of the present disclosure have been hereinabove described in detail with reference to the detailed description and the accompanying drawings, the present disclosure is not limited to the disclosed embodiments, and many substitutions, modifications and changes may be made by those skilled in the art without departing from the technical aspects of the present disclosure. Therefore, the scope of the present disclosure should not be limited to the disclosed embodiments, and should be interpreted as including the appended claims and their equivalents.

What is claimed is:

1. An insulin syringe replacement system for an insulin pump, comprising:
a control unit configured to control an operation of the insulin pump;
an actuation unit configured to perform an initialization setting operation when replacing an insulin syringe under the control of the control unit;
a battery unit configured to supply power to the control unit and the actuation unit;
a housing unit configured to mount the insulin syringe; and
a sensor unit configured to detect whether a cap of the insulin syringe mounted in the housing unit is coupled and notify it to the control unit,
wherein the insulin syringe that is mounted in the housing unit comprises a cylinder to store insulin, an outlet positioned at an end of the cylinder to dispense the insulin, a cap coupled to the outlet to prevent the insulin stored in the cylinder from leaking, a plunger gasket located at an opposite end of the cylinder to change an internal pressure of the cylinder, and a plunger movement rod to push the plunger gasket to move the plunger gasket forward,
wherein the initialization setting operation performed by the actuation unit is an operation in which the actuation unit moves the plunger movement rod forward until the plunger movement rod reaches the plunger gasket in a state that the cap is coupled to the outlet of the insulin syringe, and
wherein the control unit controls the actuation unit to perform the initialization setting operation only after receiving coupling notification of the cap of the insulin syringe mounted in the housing unit from the sensor unit, and recognizes an amount of insulin contained in the cylinder based on a movement distance or a location of the plunger movement rod after completion of the initialization setting operation performed by the actuation unit.

2. The insulin syringe replacement system for the insulin pump according to claim 1,
wherein the housing unit comprises a holder where the outlet of the insulin syringe mounted in the housing unit is situated and exposed to an outside of the insulin pump to allow the coupling of the cap,
wherein the sensor unit is a magnetic sensor and is located at the holder to detect the coupling of the outlet and the cap, and
wherein the cap that is coupled to the outlet has a magnetic property, and when the cap is coupled to the outlet, the magnetic sensor detects magnetic field lines of the cap and notifies it to the control unit.

3. The insulin syringe replacement system for the insulin pump according to claim 2,
wherein the cap that is coupled to the outlet has a coupling groove extending from a hole into which the outlet is inserted when coupled, and has a magnetic body attached to an area of a surface facing the magnetic sensor when the coupling with the outlet is normally completed, and
wherein the outlet that is coupled with the cap has a coupling protrusion on a side, the coupling protrusion configured to be engaged with the coupling groove of the cap that is coupled to the outlet, and when the normal coupling of the outlet and the cap is done, the area of the surface of the cap facing the magnetic sensor is defined.
